(19) United States
(12) Patent Application Publication
(10) Pub. No.: US 2017/0188623 A1
(43) Pub. Date: Jul. 6, 2017

(54) Method Of Manufacturing Standardized Cannabis Cigarettes

(71) Applicant: Jason Cranford, South Park, CO (US)

(72) Inventor: Jason Cranford, South Park, CO (US)

(21) Appl. No.: 15/360,989

(22) Filed: Nov. 23, 2016

Related U.S. Application Data
(60) Provisional application No. 62/258,972, filed on Nov. 23, 2015.
(60) Provisional application No. 62/280,977, filed on Jan. 20, 2016.
(60) Provisional application No. 62/258,972, filed on Nov. 23, 2015.

(51) Int. Cl. A24C 5/06; A24B 7/04; A24B 15/16; A24B 3/16; A24C 5/47; A61K 31/352; A24B 3/04

(52) U.S. Cl. CPC A24C 5/06; A61K 31/352; A24B 7/04; A24B 3/04; A24B 3/16; A24C 5/474; A24B 15/16

(57) ABSTRACT

A method of manufacturing standardized cannabis cigarettes that may comprise drying harvested cannabis plants at room temperature with a humidity of between approximately 45% and 75%; plant material, including at least one of stems and seeds, may be removed to produce a refined cannabis. The method may further include chopping the refined cannabis into cannabis particles with a median value of particle area between approximately 1.5 mm² and approximately 4.5 mm². The cannabis particles may be homogenized to produce a blended cannabis. One or more cannabis contact points of a commercial cigarette rolling machine may then be lubricated to preventing the adherence of cannabis resin to the cannabis contact points. The method may further include packing the blended cannabis into a paper rolling tube at a packing pressure of approximately 40 psi to approximately 120 psi to form the standardized cannabis cigarette assuring the controlled delivery of cannabinoids during combustion.

### CROSS-REFERENCE TO RELATED APPLICATIONS

The present application claims priority to both U.S. Provisional Application No. 62/258,972 filed on Nov. 23, 2015, titled “Manufacture Of Filtered Cannabis Cigarettes”; and U.S. Provisional Application No. 62/280,977 filed on Jan. 20, 2016, titled “A Method Of Manufacturing Standardized Cannabis Cigarettes”; the entire contents of both being expressly incorporated herein by reference.

### FIELD OF THE INVENTION

The present disclosure generally relates to the standardized manufacture of cannabis cigarettes. More particularly, the disclosure relates to the specialized processing of cannabis and adapting commercial rolling machines to manufacture cannabis cigarettes.

### BACKGROUND

Legal statutes now permit both the recreational and medical use of cannabis for the delivery of THC (Δ9-tetrahydrocannabinol), CBD (cannabidiol), and other cannabinoids to a user. Inhaling combusted cannabis is the most common, effective, and least expensive method for delivering a pharmacological action to the brain and body. The THC dose of inhaled cannabis smoke is largely determined by the concentration of precursor THCA (tetrahydrocannabinolic acid) present in the buds and small leaves and its temperature-dependent conversion to THC during combustion. In addition, state statutes may authorize low-THC high-CBD marijuana products within a narrow dosage window to treat specific illnesses or symptoms.

One convenient method for smoking cannabis is to hand-roll loose cannabis stock into a cigarette. Unfortunately, the resulting ‘joints’ have uneven burn characteristics, are harsh to the lungs, and deliver an unpredictable dose of cannabinoids. Cannabinoid dosing will depend on how loose the roll is, the size of cannabis particles, puff volume, and how uniformly cannabinoids are distributed throughout the cannabis product. Additionally, the smoldering cigarette may shed burning coals, causing a fire hazard.

Alternatively, a personal, table-top cigarette rolling device, costing as little as $20, may deliver a greater and more consistent packing density than obtainable by hand rolling, and may include a filter to reduce the harshness of the inhaled smoke. However, packing density and burn characteristics will still depend upon the particle sizes of loose cannabis, which may range from dust-sized to a particle length greater than the cigarette diameter; upon the humidity content, which may vary with the local climate and storage environment; and upon the skills of the roller and the limitations of the machine.

Rolling equipment designed for tobacco may be utilized to attempt to roll uniform cannabis cigarettes. Unfortunately, cannabis has different handling and chemical properties (e.g. sticky resin) than tobacco, and the delivery objectives of cannabis are different from tobacco's flavor, additives, and nicotine. Tobacco devices are not likely to efficiently deliver the recreational and medical objectives of a cannabis cigarette without modification.

Additionally, home cultivators may grow and harvest high quality cannabis. However, the cultivation process may involve the use of pesticides and herbicides to reduce costs but which are dangerous if inhaled. Also, the drying period may be too short or may occur in uncontrolled environments, resulting in crumbling buds and leaves and/or variable moisture content, which may lead to unevenness in packing density and cannabinoid distribution.

As may be appreciated, there exists a need in the art for a standardized manufacture of safe cannabis cigarettes having uniform burn characteristics and a controlled, predictable delivery of cannabinoids (dosing). Also, there exists a need in the art for standardized cannabis cigarettes which are easy to handle and as neat as commercially rolled tobacco cigarettes. Further, there exists a need in the art for cannabis cigarettes with reduced heat and particulates entering the lungs; as well as a need in the art for cannabis cigarettes free of dangerous chemicals, such as the burn retardants used in tobacco cigarettes.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In one embodiment, a method of manufacturing standardized cannabis cigarettes is disclosed having a controlled delivery of cannabinoids; the method comprises drying harvested cannabis plants at room temperature to a humidity of between approximately 45% and 75% to form dried cannabis. This method may further include removing unsuitable plant material from the dried cannabis to produce a refined cannabis, the unsuitable plant material including at least one of stems and seeds. The method may further include chopping the refined cannabis into cannabis particles such that a particle area of the cannabis particles has a median value of between approximately 1.5 mm² and approximately 4.5 mm². The method may further include homogenizing the cannabis particles to produce an evenly blended cannabis. The method may further include lubricating one or more cannabis contact points of a commercial cigarette rolling machine with a lubricant for preventing the adherence of cannabis resin to the cannabis contact points. The method may further include packing the blended cannabis into a paper rolling tube, the rolling machine exerting a packing pressure of approximately 40 psi to approximately 120 psi to form the standardized cannabis cigarette assuring the controlled delivery of cannabinoids during combustion.

In another embodiment, there is disclosed a filtered standardized cannabis cigarette comprising a volume of blended cannabis made of cannabis particles with a particle area having a median value of between approximately 1.5 mm² and approximately 4.5 mm². The blended cannabis may be prepared from harvested cannabis plants dried at room temperature at a humidity of between approximately 45% and approximately 75%. A paper rolling tube may be tipped with a filter on one end and may enclose the volume of blended cannabis, the filter for removing at least one of particulates and heat. The blended cannabis may be packed into the paper rolling tube at a packing pressure of between approximately 40 and approximately 120 psi for assuring the controlled delivery of cannabinoids during combustion.

### BRIEF DESCRIPTION OF THE DRAWINGS

The above, and other, aspects, features, and advantages of several embodiments of the present disclosure will be more apparent from the following Detailed Description as presented in conjunction with the following several figures of the Drawing.

FIG. 1 illustrates a process flow for manufacturing cannabis cigarettes, in accordance with an embodiment of the present disclosure.

FIG. 2 illustrates a cannabis cigarette as an article of manufacture, in accordance with an embodiment of the present disclosure.

FIG. 3 illustrates a cumulative distribution probability for measured particle area of cannabis particles within a standardized cannabis cigarette, in accordance with an embodiment of the present disclosure.

FIG. 4 illustrates a histogram of measured passive smoldering rates for tobacco and cannabis cigarettes, in accordance with an embodiment of the present disclosure.

FIG. 5 illustrates a commercial rolling machine for rolling cannabis cigarettes, in accordance with an embodiment of the present disclosure.

Corresponding reference characters indicate corresponding components throughout the several figures of the Drawings. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common but well-understood elements that are useful or necessary in commercially feasible embodiment are often not depicted in order to facilitate a less obstructed view of these various embodiments of the present disclosure.

### REFERENCE NUMERALS

10 Method For Manufacturing Standardized Cannabis Cigarettes
12 Cigarette Length
14 Filter Length
16 Combustible Length
20 Homogenized Cannabis
22 Filter
23 Filter Inside End
24 Paper Rolling Tube
26 Cigarette Diameter
30 Drying Process
32 Dried Cannabis
34 Sorting Process
36 Refined Cannabis
38 Stems And Seeds
40 Chopping Process
42 Cannabis Particles
44 Sifting Process
46 Sifted Cannabis
48 Mixing Process
49 Large Particles
50 Blended Cannabis
51 Volume Of Blended Cannabis
52 Post-Blend Humidification Process
54 Lubricating Process
56 Rolling And Packing Process
58 Storing Process
60 Rolling Machine
62 Blanks Cartridge
64 Filler Funnel
66 Discharge Chute
68 Packing Piston
80 Particle Area Distribution
90 Burn Rate Histogram
100 Cannabis Cigarette

### DETAILED DESCRIPTION

The following description is not to be taken in a limiting sense, but is made merely for the purpose of describing the general principles of exemplary embodiments; many additional embodiments of this disclosure are possible. It is understood that no limitation of the scope of the disclosure is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but does not necessarily, all refer to the same embodiment.

Further, the described features, structures, or characteristics of the present disclosure may be combined in any suitable manner in one or more embodiments. In the Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure. Any alterations and further modifications in the illustrated devices, and such further application of the principles of the disclosure as illustrated herein are contemplated as would normally occur to one skilled in the art to which the disclosure relates.

Unless otherwise indicated, the drawings are intended to be read (e.g., arrangement of parts, proportion, degree, etc.) together with the specification, and are to be considered a portion of the entire written description of this disclosure. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate. Also, as used herein, terms such as “positioned on” or “supported on” mean positioned or supported on but not necessarily in direct contact with the surface.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

Further, all numbers expressing dimensions, physical characteristics, and so forth, used in the specification and claims are to be understood as being modified in all instances by the term “about”. Accordingly, unless indicated to the contrary, the numerical values set forth in the following specification and claims can vary depending upon the desired properties sought to be obtained by the practice of the disclosure. At the very least, and not as an attempt to limit the application of the doctrine of equivalents to the scope of the claims; each numerical parameter should at least be construed in light of the number of reported significant digits and by applying ordinary rounding techniques. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.3, or 5.5 to 10, or 2.7 to 6.1.

For the purposes of promoting an understanding of the principles of the present disclosure, reference will now be made to the embodiments illustrated in the drawings and specific language will be used to describe the same.

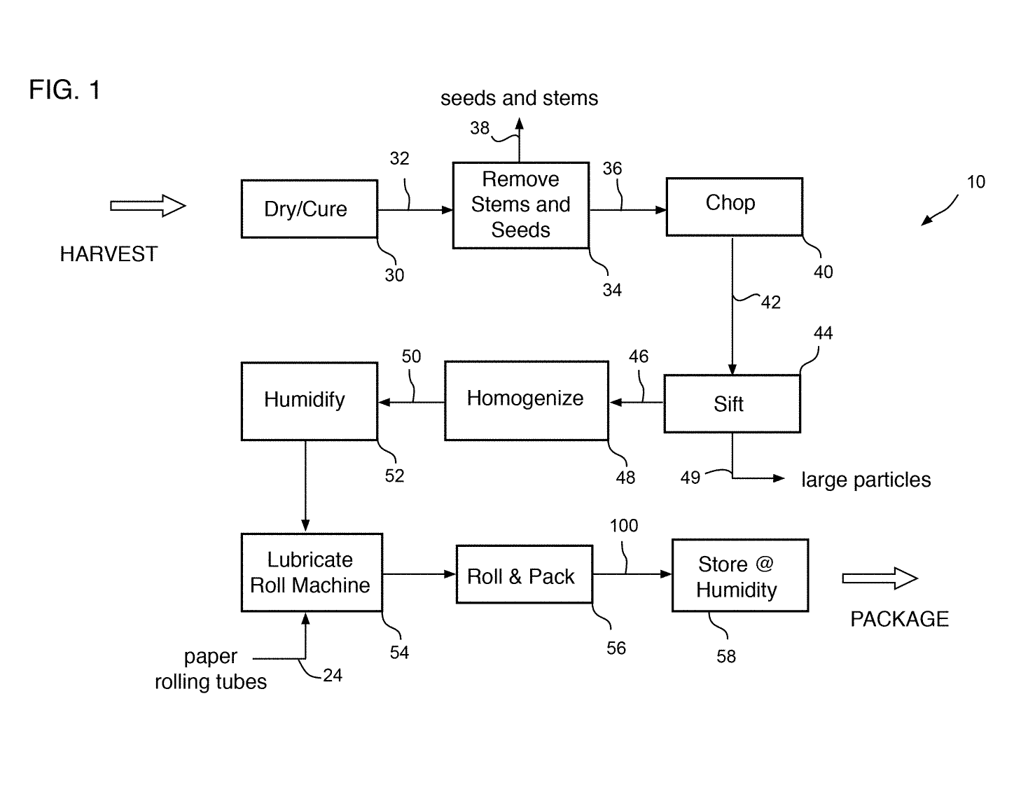

Referring to FIG. 1, in various embodiments, a method for manufacturing 10 may comprise harvesting cannabis strains of high THCA content and drying 30 the harvested cannabis plants at room temperature with a humidity of between approximately 45% and approximately 75%, resulting in dried cannabis 32. For example, room temperature may range from approximately 21° C. to approximately 27° C. The THCA content in the dried cannabis 32 may be selected to lie between approximately 8% and approximately 14% by weight. The plants may be inverted during drying. Drying time under controlled-humidity conditions may be range from approximately 8 to approximately 16 days.

Continuing with FIG. 1, in the preferred embodiment, THCA content in the dried cannabis 32 may range from approximately 10% to approximately 11% by weight. Room temperature during drying may be kept at approximately 24° C. The relative humidity during the drying process may be held at approximately 60%, and may range from 50% to 70%. In this embodiment, drying time may be approximately 11 to approximately 13 days.

The harvested cannabis may be organically grown without pesticides or added chemicals. The plants growth cycle may last approximately 120 days before harvesting and drying. Plant growing time may vary by a month or more, depending on climate, irrigation, or hydroponics conditions. Drying 30 may further include a curing process to dissipate harshness in the smoked cannabis where curing may include putting the dried cannabis in an airtight container for a matter of days or weeks.

Continuing, in various embodiments, cannabis strains may be selected for concentrations of cannabinoids besides high THCA. For example, cannabis strains having a low THCA content (e.g. <1%) and a high CBD content (e.g. >10%) may be selected for medicinal applications or for pain relief. Alternately, THCA content may be specified in terms of milligrams (mg) instead or % weight. Advantageously, the prescribed drying process of 45-75% humidity at room temperature over a period of 8-16 days may result in evenly distributing the cannabinoids throughout the leaves, buds, and trichomes of the cannabis plant for providing a controlled delivery of cannabinoids upon combustion. Also, the prescribed humidified drying process 30 may establish good handling characteristics for the succeeding steps in manufacturing such as uniform burn characteristics and reduced breakage of leaf and buds.

Referring still to FIG. 1, in various embodiments, unsuitable plant material 38, including at least one of stems and seeds, may be removed or sorted 34 from the dried cannabis 32, leaving a refined cannabis 36 comprising plant buds, trichomes, and/or small leaves (<3 cm). The unsuitable plant material 38 may contain few cannabinoids, may contribute to a harshness when smoked, and/or may not support a uniform cigarette burn characteristic. Refined cannabis 36 may then be chopped 40 into cannabis particles 42 such that a particle area of the cannabis particles 42 has a median value of between approximately 1.5 mm² and approximately 4.5 mm². The chopping operation 40 may be performed with a rotating blade within a food processor (not shown) rotating for between approximately 3 and approximately 10 minutes. In a preferred embodiment, the rotating blade may chop the refined cannabis 36 for between approximately 5 and approximately 7 minutes in order to get a distribution of particle area that enables good packing density within a cigarette and uniform burn characteristics. Alternatively, in an embodiment not shown, refined cannabis 36 may be chopped 40 by hand with a knife or flat blade.

Referring still to FIG. 1, in an embodiment, cannabis particles 42 may be sifted 44 after chopping 40 in order to remove large particles 49 whose particle diameter is larger than approximately 10 mm, thereby producing sifted cannabis 46. A mesh screen (not shown) may be used to sift out particles larger than approximately 10 mm, leaving particles of different sizes and shapes. The mesh screen may be made of stainless steel, plastic, or composite, and may be square in its aperture similar to conventional window screen. Particles larger than 10 mm may be restrained by the screen while the desired sifted cannabis 46 may pass through the screen for further processing. Particle shape may be typically an irregular rectangle or square and have a length and a width (not shown). The particle diameter may be the longer of two orthogonal dimensions of a randomly selected length and width. Particle area may be the product of a randomly selected length and width. Alternately, the particle diameter may be the average diameter of a roundish particle.

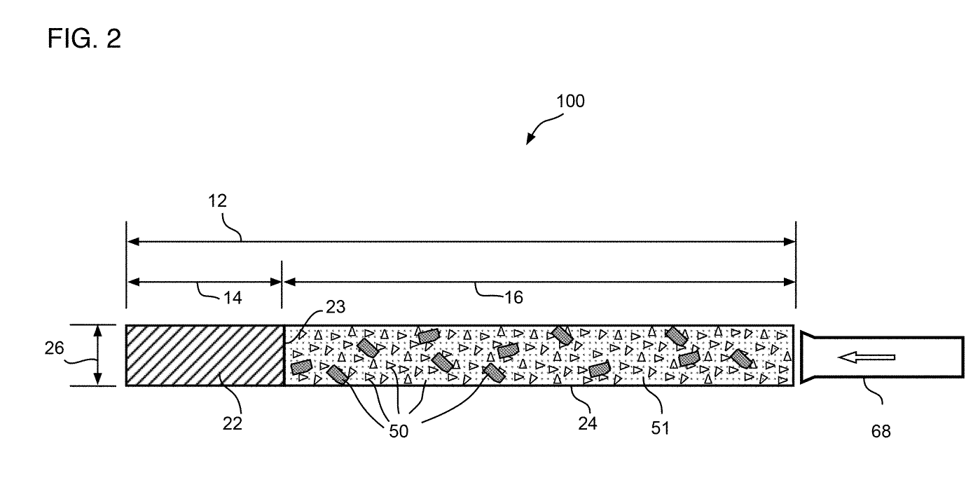

Referring to FIG. 2, since cigarette diameter 26 of cannabis cigarette 100 may be nominally 8 mm and may range from 7 mm to 10 mm, sifting out approximately 10 mm particles may prevent restrictions to packing cannabis into a paper rolling tube 24. Alternately, an aperture size of a mesh screen for sifting operation 44 may be sized to block cannabis particles 42 larger than approximately the inside diameter of the paper rolling tube receiving packed cannabis within a cannabis cigarette 100. Alternatively, cannabis particles 42 may not be sifted if there are few or no particles large enough to impair the efficiency of processing downstream of the chopping operation 40.

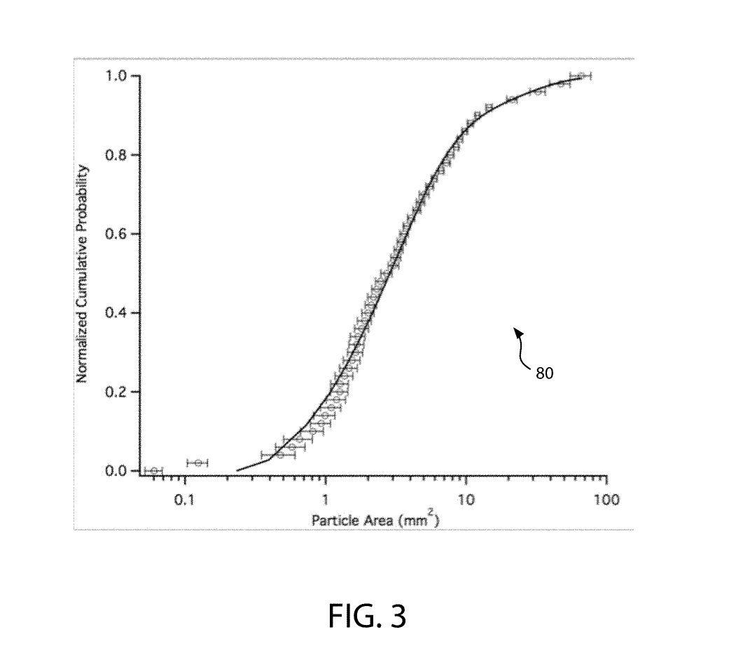

Referring now to FIGS. 1 and 3, in various embodiments, particle area distribution 80 (FIG. 3) after chopping 40 and sifting 44 operations may plot particle areas which range from approximately 0.04 mm² to approximately 90 mm². If the particles are square in shape, then a largest sifted particle of approximately 10 mm on both sides may possess a particle area of approximately 100 mm². A median value for particle area in the sifted cannabis 46 may be approximately 3 mm², as shown in FIG. 3, where 50% of the cigarette filler particles had an area between 0.04 mm² and 3 mm² and 50% had an area of between 3 mm² and 90 mm²; and 90% of the sifted cannabis filler may lie between approximately 0.5 mm² and approximately 25 mm². Advantageously, a median particle area of 1.5 mm² to 4.5 mm² may represent a finer cut of cannabis filler than hand-rolled ‘joints’ and may produce a consistent and high cannabinoid conversion efficiency per cigarette unobtainable with current techniques.

The effect of a fine cut such as a median particle area of from approximately 1.5 mm² to approximately 4.5 mm² and a relatively uniform particle area distribution, for example approximately 90% of the particle area being between 0.5 mm² and 25 mm², may beneficially influence the aerosol and chemistry of the burn for producing a controlled and efficient cannabinoid delivery. The distribution of particle size shown in FIG. 3 may also provide a stably interlocking blend of cannabis particles which, when packed, may deliver uniform combustion properties and controlled cannabinoid delivery.

Referring now to FIGS. 1 and 2, in various embodiments, sifted cannabis 46 may be followed by a homogenizing process 48 to mix the particles and produce a blended cannabis 50. Homogenizing 48 may be a simple mixing step following the chopping (and sifting, if applicable) operation such that the distribution of particle areas is approximately the same for each cigarette that is rolled. For example, the step 48 of homogenizing may distribute particles according the median and 80% distribution of particle area specified for chopping step 40 for any randomly selected volume of blended cannabis sized to fill a cannabis cigarette in the subsequent rolling and packing step. Homogenizing 48 may occur naturally within the chopping process 40 of the rotating-blade food processor and no separate homogenizing device may need to be employed. In an embodiment, a batch cement-style mixer may be used to homogenize 48 the cannabis particles (42 or 46). The cement-style mixer may be a beveled or tilted bin that is rotated in a manner similar to a cement mixer to make the particle distribution uniform. For example, the mixer may rotate the cannabis particles at approximately 20 rpm (revolutions per minute) for approximately 1 hour. Mixing may occur dry, or may occur wet followed by a drying process (not shown). Additionally, the homogenizing may create a bonding or an interlocking between adjacent particles such that uniformity of packing density is achieved.

Referring to FIGS. 1, 2, and 5, in a preferred embodiment, the batch mixer may have a batch size of approximately 11 kg. Alternately, batch size in homogenizing process 48 may range from a few kilograms to 100 kilograms, depending on the scale of the manufacturing operation. Also, mixing (homogenizing) may be performed by hand by shaking or agitating a container following chopping process 40. Homogenizing may also occur naturally as blended cannabis is poured into a filler funnel 64 of the rolling machine 60.

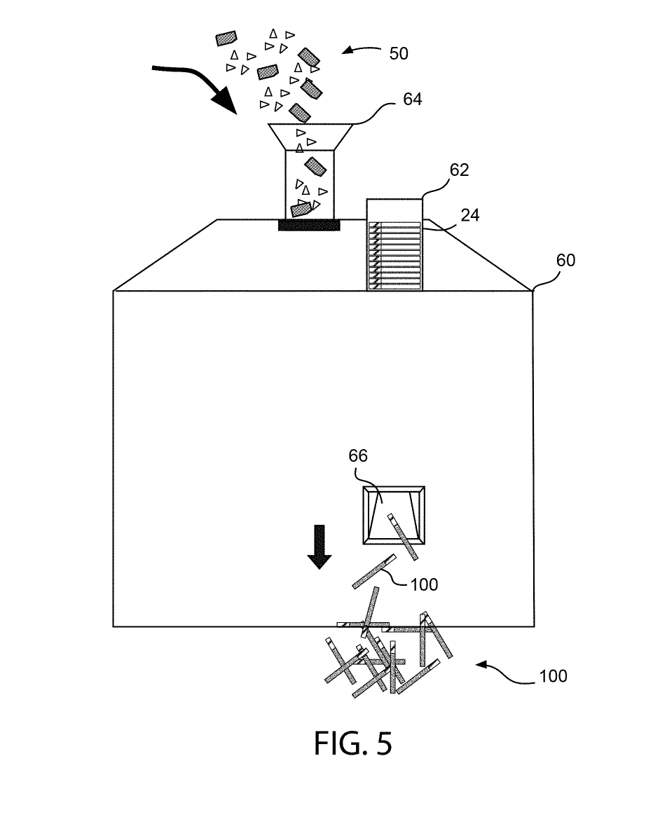

Continuing with FIG. 1, in various embodiments, following homogenization step 48, the blended cannabis 50 may be humidified 52 in the presence of circulating fans (not shown) before rolling and packing step 56 at rolling machine 60 (FIG. 5), the humidifying 52 for at least one of making more uniform the packing density of cannabis cigarette 100 and avoiding the breakage of cannabis particles. For example, cannabis particles may become dry during chopping and other manufacturing steps, and the particles may break, creating a dust that does not pack well into a paper rolling tube 24. The humidifying step 52 may be performed to prevent particle breakage and may occur at a room-temperature humidity of between approximately 45% and approximately 75%. The blended cannabis 50 may be rotated or inverted into another container periodically to prevent the growth of fungi. For example, a storage container of blended cannabis 50 being humidified may be poured into an empty container approximately every 12 hours to prevent the growth of fungi. The humidifying process 52 may last from approximately 1 to approximately 4 days. Alternatively, humidification 52 may be continued for approximately 2 to 3 days.

Referring now to FIGS. 1, 2, and 5, in various embodiments, one or more cannabis contact points (not shown) of a commercial cigarette rolling machine 60 may be lubricated 54 with a lubricant (not shown) for preventing the adherence of cannabis resin to the cannabis contact points. The lubricant may be a food-grade lubricant, and the food-grade lubricant may comprise at least one the following food-grade oils: vegetable, canola, soy, hemp, and corn. Advantageously, lubricating one or more contact points within rolling machine 60 may effectively adapt a commercial tobacco rolling machine for use with cannabis. The lubricant may be hand sprayed onto the contact points, wiped, or otherwise applied. The one or more contact points may include one or more of a trough for holding blended cannabis waiting to be loaded into the paper rolling tube 24, the packing piston 68, a filler funnel 64, any passageways passing cannabis through the rolling machine 60, a cigarette discharge chute 66, and any surfaces handling packed cigarettes 100.

Referring still to FIGS. 1, 2, and 5, in various embodiments, the lubricant may be any lubricant whose residue is minimal and non-toxic such that any cannabis contacting the lubricant is safe to combust and inhale. Paper rolling tubes 24 may be loaded into rolling machine 60, and a blanks cartridge 62 loadable into rolling machine 60 may hold a quantity of rolling tubes 24 for supplying rolling machine 60. Blended cannabis 50 having a distribution of particle sizes (FIG. 3) according to chopping step 40 may be loaded into rolling machine 60 for packaging into cannabis cigarettes 100 exiting discharge chute 66. The blended cannabis 50 may be packed 56 into a paper rolling tube 24 by rolling machine 60 exerting a packing pressure of approximately 40 psi to approximately 120 psi to form the cannabis cigarette 100 assuring the controlled delivery of cannabinoids during combustion. In an embodiment, a packing piston 68 (FIG. 2) of rolling machine 60 may exert the packing pressure on a volume of blended cannabis positioned to fill rolling tube 24 over a combustible length 16. In an embodiment, the packing piston 68 may be made of Teflon™ for preventing the adherence of cannabis resin thereto.

Continuing with FIGS. 1, 2, and 5, in various embodiments, the packing pressure may be approximately 80 psi (pounds per square inch), and may for example range from approximately 60 psi to approximately 100 psi. The rolling machine 60 may be designed by RYO Machines LLC. Alternately, any commercial rolling machine adapted appropriately with the above modifications of lubricant, drying process, chopping and sifting processes, and post-sift humidification may be adapted to manufacture standardized cannabis cigarettes having a controlled delivery of cannabinoids. The cannabis cigarette 100 of cigarette length 12 may be tipped with a filter 22 having filter length 14 (FIG. 2) for removing at least one of particulates and heat drawn from combusting cannabis. The filter may be included as part of paper rolling tube 24 so that the packing process pushes blended cannabis 50 against the inside end 23 of filter 22.

Continuing still with FIGS. 1, 2, and 5, in various embodiments, filter 22 may be based on a cellulose acetate or a cotton material. In an embodiment, a custom filling spout (not shown) may be inserted into the paper rolling tube 24 within rolling machine 60 to fill tube 24 with blended cannabis 50 at a packing pressure of approximately 80 psi.

In an embodiment, referring to FIGS. 1 and 2, cannabis cigarettes 100 may be stored 58 in a humidity controlled environment from several hours to several days for stabilizing the controlled delivery of cannabinoids. For example, cannabis cigarettes 100 may be stored 58 after packing at a humidity of between approximately 20% and approximately 40% for stabilizing the controlled delivery of cannabinoids. In an embodiment, cannabis cigarettes may be collected in shallow trays (e.g. at approximately 0.1 meters deep) and stored at approximately 30% humidity at room temperature for approximately 12 hours before packaging multiple cigarettes into a shipping package (not shown). A shipping package may hold approximately 10 cannabis cigarettes. It is understood that the storage humidity (e.g. 30%), tray depth (e.g. 0.1 m), and storage time (e.g. approximately 12 hours) are interdependent and may be adjusted to other values to optimize the manufactured cigarettes of various cannabinoid content. For example, a shallower storage tray may only require a 6 hour storage time, a lower humidity setting may require a 24 hour storage time, and a high-CBD content cigarette may require a deeper or shallower storage tray.

Referring now to FIG. 2, in various embodiments, filtered standardized cannabis cigarette 100 may comprise a volume 51 of blended cannabis 50 made of cannabis particles having a particle area with a median value of between approximately 1.5 mm² and approximately 4.5 mm² filling a paper rolling tube 24 which may be tipped with a filter 22 on one end of the tube 24. The filter 22 may be for removing at least one of particulates and heat, so as to reduce the harshness of the smoke resulting from combustion. The blended cannabis 50 may be prepared from harvested cannabis plants dried at room temperature at a humidity of between approximately 45% and approximately 60%. The volume 51 of cannabis may be packed into rolling tube 24 at a packing pressure of between approximately 40 and approximately 120 psi for assuring the controlled delivery of cannabinoids during combustion. Packing piston 68 may be used to apply a packing pressure of approximately 80 psi (pounds per square inch), and may for example range from approximately 60 psi to approximately 100 psi.

Continuing with FIG. 2, in various embodiments, a cigarette length 12 of cannabis cigarette 100 may range from approximately 70 mm to approximately 120 mm. Preferably, cigarette length 12 may be approximately 82 mm including a 12 mm filter, or may be approximately a ‘king size’ 84 mm in length, and may have a cigarette diameter 26 of approximately 8 mm. The cigarette 100 may also have a length 12 of approximately 100 mm or a length 12 of approximately 120 mm long. In an embodiment, the THCA (tetrahydrocannabinolic acid) content in the dried cannabis 32 may be selected to be between approximately 8% and approximately 14% by weight. For example, the THCA content in the dried cannabis 32 may range from approximately 10% to approximately 11% by weight. In an embodiment, the harvested cannabis for cigarette 100 may have been prepared by a drying process occurring at approximately 60% humidity over approximately 8 to approximately 16 days. The distribution of particle size in the volume 51 of blended cannabis may place approximately 80% of the particle areas between approximately 0.3 mm² and approximately 20 mm².

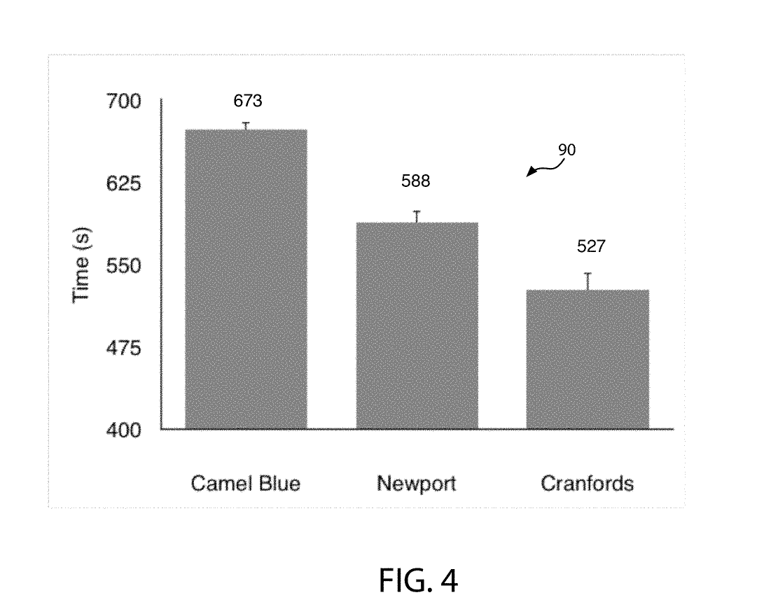

Referring now to FIG. 4, in various embodiments, an inverted burn time histogram 90 may illustrate passive smoldering results for Cranfords cannabis cigarettes and for Camel Blue and Newport tobacco cigarettes. The test was performed to measure burn rate and consistency by hanging an 82 mm by the filter region in an open Ball® 16 oz canning jar immediately after igniting the cigarette for a period of 10-11 seconds. The smoldering coal for each cigarette was allowed to burn to a demarcated point where 5 cm of the cigarette was converted to ash with ambient temperature between 22-26° C. and a relative humidity of 20% and the smoldering rate was measured using a digital stopwatch.

Continuing with FIG. 4, the disclosed method of manufacturing a cannabis cigarette provides approximately the same burn rate as a tobacco cigarette: the average burn rate for the Cranfords cannabis cigarette may be only approximately 16% faster than the burn rate for the average Camel and Newport tobacco cigarettes. Also, the standard error of the mean of burn duration shown in FIG. 4 may be less than approximately 100 seconds, as indicated by the error bars at the top of each histogram. In another embodiment (not shown), the standard error of the mean of burn duration for a Cranfords cigarette may be less may be approximately 50 seconds. The cannabis cigarettes of the present disclosure may have a faster burn rate than tobacco cigarettes because of tobacco additives like humectants which retard the smoldering rate. Advantageously, the disclosed cannabis cigarette and manufacturing method may therefore have a uniform burn characteristic from cigarette to cigarette and may have a slow, controlled burn rate, thereby enabling a controlled, predictable delivery of cannabinoids (dosing). The resulting controlled delivery of cannabinoids may make possible the targeting of a narrow and effective window required particularly for therapeutic and medical usage of cannabis.

Continuing, in various embodiments, controlled delivery may mean that a high THC cannabis cigarette manufactured according to disclosed process may deliver a high dosage of THC per inhalation and a low-THC cannabis may deliver a low dosage of THC per inhalation. In contrast, an uncontrolled or inconsistent delivery of cannabinoids from a cannabis cigarette would begin to blur the boundaries between high-THC cannabis and low-THC cannabis, and may thereby ruin the desired recreational or medicinal benefit of cannabis consumption, and may impair cognitive function, mood, and judgment.

Information as herein shown and described in detail is fully capable of attaining the above-described object of the present disclosure, the presently preferred embodiment of the present disclosure; and is, thus, representative of the subject matter; which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

What is claimed is:

1. A method of manufacturing standardized cannabis cigarettes having a controlled delivery of cannabinoids, the method comprising:
drying harvested cannabis plants to form a dried cannabis;
removing unsuitable plant material from the dried cannabis to produce a refined cannabis, the unsuitable plant material including at least one of stems and seeds;
chopping the refined cannabis into cannabis particles such that a particle area of the cannabis particles has a median value of between approximately 1.5 mm² and approximately 4.5 mm²;
homogenizing the cannabis particles to produce a blended cannabis;
lubricating one or more cannabis contact points of a commercial cigarette rolling machine with a lubricant for preventing the adherence of cannabis resin to the cannabis contact points; and
packing the blended cannabis into a paper rolling tube, the rolling machine exerting a packing pressure of approximately 40 psi to approximately 120 psi to form the standardized cannabis cigarette assuring the controlled delivery of cannabinoids during combustion.

2. The method of claim 1, further comprising:
selecting a THCA (tetrahydrocannabinolic acid) content in the harvested cannabis to be above 0.3% by weight.

16. The method of claim 1, wherein:
the drying occurs at approximately 60% humidity over approximately 8 to approximately 16 days.

17. The method of claim 1, further comprising:
sifting cannabis particles after the chopping by using a mesh screen to remove particles whose particle diameter is larger than approximately 10 mm, thereby producing sifted cannabis.

18. The method of claim 4, wherein:
approximately 90% of the sifted cannabis has a particle area of between approximately 0.5 mm² and approximately 25 mm².

19. The method of claim 1, wherein:
the chopping operation uses a rotating blade within a food processor to chop the refined cannabis for between approximately 3 and approximately 10 minutes.

20. The method of claim 1, wherein:
a batch cement-style mixer is used to homogenize the cannabis particles.

21. The method of claim 7, wherein:
where the mixer rotates the cannabis particles at approximately 20 rpm for approximately one hour.

22. The method of claim 1, further comprising:
humidifying the blended cannabis in the presence of circulating fans after homogenizing and before sending the cannabis to the rolling machine, the humidifying for at least one of making more uniform the packing pressure and avoiding particle breakage.

23. The method of claim 1, wherein:
the lubricant comprises at least one the following food-grade oils: vegetable, canola, soy, hemp, and corn.

24. The method of claim 1, wherein:
the rolling machine is designed by RYO Machines LLC.

25. The method of claim 1, wherein:
the packing pressure is approximately 80 psi.

26. The method of claim 1, further comprising:
a packing piston exerting the packing pressure and being made of Teflon™ for preventing the adherence of cannabis resin to the packing piston.

27. The method of claim 1, further comprising:
tipping the cannabis cigarette with a filter at one end of the paper tube.

28. The method of claim 1, further comprising:
storing the cannabis cigarettes after packing at a humidity of between approximately 20% and approximately 40% for stabilizing the controlled delivery of cannabinoids.

16. A filtered standardized cannabis cigarette, comprising:
a volume of blended cannabis made of cannabis particles with a particle area having a median value of between approximately 1.5 mm² and approximately 4.5 mm², the blended cannabis having been prepared from harvested and dried cannabis plants;
a paper rolling tube tipped with a filter on one end of the tube, the filter for removing at least one of particulates and heat, the paper rolling tube enclosing the volume of blended cannabis; and
where the blended cannabis is packed into the paper rolling tube at a packing pressure of between approximately 40 and approximately 120 psi for assuring the controlled delivery of cannabinoids during combustion.

18. The cannabis cigarette of claim 16, wherein:
a cigarette length of the cannabis cigarette is between approximately 70 mm and approximately 120 mm.

21. The cannabis cigarette of claim 16, wherein:
a THCA (tetrahydrocannabinolic acid) content in the harvested cannabis is selected to be above 0.3% by weight.

22. The cannabis cigarette of claim 16, further comprising:
the blended cannabis having particles larger than a particle diameter of approximately 10 mm removed by sifting, leaving approximately 90% of the blended cannabis particles having a particle area of between approximately 0.5 mm² and approximately 25 mm².

23. The cannabis cigarette of claim 16, wherein:
the packing pressure is approximately 80 psi.
